(19) United States
(12) Patent Application Publication
(10) Pub. No.: US 2003/0091666 A1
(43) Pub. Date: May 15, 2003

(54) Methods and compositions for treating dermatological disorders with Morinda citrifolia

(76) Inventor: Howard Murad, Marina del Rey, CA (US)

Correspondence Address: PENNIE & EDMONDS LLP, 1667 K STREET NW, SUITE 1000, WASHINGTON, DC, 20006

(21) Appl. No.: 09/987,354

(22) Filed: Nov. 14, 2001

(51) Int. Cl. A61K 35/78; A61K 38/48; A61K 31/7024

(52) U.S. Cl. 424/765; 424/94.64; 514/23; 514/557; 424/769

(57) ABSTRACT

This application relates to methods and pharmaceutical compositions for treating dermatological disorders. The methods include administering a therapeutically effective amount of an extract of Morinda citrifolia. The compositions include an extract of Morinda citrifolia; and at least one of a moisturizing agent in an amount sufficient to facilitate hydration of the skin or hydrogen peroxide in an amount sufficient to cleanse at least a portion of the skin.

### TECHNICAL FIELD

The invention relates to pharmaceutical agents containing an extract of Morinda citrifolia and at least one of a moisturizing agent or hydrogen peroxide and to methods of using an extract of Morinda citrifolia to treat dermatological disorders.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The skin is the most environmentally-stressed organ in mammals, particularly in humans. The skin is exposed to a wide variety of biological, chemical, and physical attacks including, for example, exposure to toxic chemicals and hostile environments and is also the only organ directly exposed to ultraviolet (“UV”) light in the presence of oxygen. [See, e.g., P. Mayer, et al., Cosmetic & Toiletries, 108:99-109 (February 1993)]. Lengthy exposure of the skin to UV light typically damages the skin, resulting in sunburn, photoaging, and carcinogenesis.

The principal functions of the skin include protection, excretion, secretion, absorption, thermoregulation, pigmentogenesis, accumulation, sensory perception, and regulation of immunologicalprocesses. These functions are detrimentallyaffectedbythe structural changes in the skin due to aging and excessive exposure to the environment and the sun. The mechanical properties of the skin, such as elasticity, are controlled by the density and geometry of the network of collagen and elastic fiber tissue therein. Damaged collagen and elastin lose their contractile properties, resulting in skin wrinkling and skin surface roughness. As the skin ages or becomes unhealthy, it acquires sags, stretch marks, bumps, bruises or wrinkles, it roughens, and it has reduced ability to synthesize Vitamin D. Aged skin also becomes thinner and has a flattened dermoepidermal interface because of the alterations in collagen, elastin, and glycosaminoglycans. [Fenske, N. A, and Lober, C. W., J. Am. Acad. Dermatol., 15:571-585 (October 1986); Montagna, W. and Carlisle, K., Journal of Investigative Dermatol., 73(1):47-53 (1979)]. The physiological changes associated with skin aging also include impairment of the barrier function and decreased turnover of epidermal cells. [Cerimele, D., et al., Br. J. Dermatol., 122 Suppl. 35, p. 13-20 (April 1990)].

Various pharmaceuticals have been used for the management of skin conditions. A variety of vitamins and minerals have individually been administered to treat certain skin and other problems that occur when the patient has a deficiency of that vitamin or mineral. Vitamin A, for example, assists in the treatment of acne and facilitates wound healing; vitamin C (ascorbic acid) assists in the prevention of skin bruising and wound healing; vitamin E is an antioxidant; and copper assists in the treatment of elastic tissue defects. [Neldner, K. H., Amer. Acad. Derm. Annl. Mtg., Washington D.C., Dec. 6, 1993]. Topical use of vitamin C is also believed to ward off sun damage, reduce breakdown of connective tissues, and possibly promote collagen synthesis. [Dial, W., Medical World News, p. 12, March 1991]. Vitamin E is used topically as an anti-inflammatory agent, for enhancement of skin moisturization, for UV-ray protection of cells, and for retardation of premature skin aging.

Certain herbs have been found helpful in protecting the skin from the environment's harmful effects. Herb extracts such as burdock root, echinac ea, yellow dock root, and grape seeds posses detoxifying properties that have been individually applied to help the body eliminate harmful free radicals. Burdock root contains the active ingredient inulin, and is useful in treating cancerous skin conditions, as well as inflammation. Echinaco side and caffeoyl derivatives present in echinacea act as antioxidants, which protect the skin when applied topically. [R. Facino, et al., Planta Med. 61:510-514 (1995)]. Yellow dock root contains the active constituent chrysarobin, which has been used in the treatment of chronic skin diseases, such as eczema, leprosy, psoriasis, and cancer. [M. Tierra, “Planetary Herbology,” p. 194 (1988)]. Catechin-based preparations, including proanthanols and proanthocyanidins, are powerful antioxidants. These compounds are found in flowers, plant leaves, and grape seeds, for example. [Lubell, A., Cosmetic Dermatol., 9(7):58 & 60 (July 1996)]. The oligomeric proanthocyanidins (OPC's) are potent bioflavanoids found in grape seeds and are thought to be potent antioxidants possessing 20 times the antioxidant power of vitamin C and 50 times the antioxidant power of vitamin E. These herbs have been individually used both topically and orally to protect the skin from various afflictions.

Fruits, vegetables, and commonly used spices and herbs have also been stated to contain cancer protective factors [L. Dragsted, M. Strube, and J. C. Larsen, Pharmacology and Toxicology, v. 72, S1, pp. S116-S135 (1993)].

Extracts from the plant Morinda citrifolia are useful for treating abrasions, atherosclerosis, boils, burns, chronic fatigue syndrome, colds, cold sores, depression, eye inflammations, gingivitis, high blood pressure, indigestion, kidney disease, menstrual cramps, respiratory disorders, skin inflammation, sprains, tumors, arthritis, bladder infections, bowel disorders, cancer, circulatory weakness congestion, constipation, diabetes, fever, gastric ulcers, headaches, immune weakness, intestinal parasites, menstrual irregularities, mouth sores, ringworn, sinusitis, thrush, and wounds [R. Elkins, Noni (Morinda Citrifolia), Woodland Publishing, Incorporated, UT, (1997). Extracts of Morinda citrifolia may also have antimicrobial activity against microorganisms related to skin infections [Mastura Mohtar, Khozirah. Shaari, N. Azah Mohdd. Ali, and Abd. Manaf Ali, J. Tropical Forest Products, 4(2):199-206, (1998)].

U.S. Pat. No. 5,288,491 to Moniz discloses a method for processing the noni plant into a powder.

U.S. Pat. No. 6,254,913 B1 to Wadsworth et al. discloses a process for extracting and purif yng the dietary fiber from the Indian mulberry (Morinda citrifolia) to provide a high fiber dietary product.

U.S. Pat. No. 6,214,351 B1 to Wadsworth et al. discloses a process for extracting and purifying the oil from the seeds of the Indian mulberry (Morinda citrifolia).

Although the above references disclose various uses of Morinda citrifolia there is no disclosure or suggestion of pharmaceutical compositions containing Morinda Citrifolia and at least one of a moisturizing agent or hydrogen peroxide or methods of employing Morinda Citrifolia for managing dermatological conditions.

### SUMMARY OF THE INVENTION

The invention relates to pharmaceutical compositions for managing a dermatological condition. The compositions comprise an extract of Morinda citrifolia and at least one of a moisturizing agent in an amount sufficient to facilitate hydration of the skin or hydrogen peroxide in an amount sufficient to cleanse the skin. The extract of Morinda citrifolia is present in an amount of from about 0.01 to 80 percent by weight of the composition, the moisturizing agent is present in an amount of about 0.01 to 20 percent by weight of the composition, and the hydrogen peroxide is present in an amount of about 0.01 to 6 percent by weight of the composition.

The moisturizing agent can be a hydrophobic moisturizing agent, such as ceramide, borage oil, tocopherol, tocopherol linoleate, dimethicone, glycerine, or a mixture thereof. The moisturizing agent can be a hydrophilic moisturizing agent, such as hyaluronic acid, sodium peroxylinecarbolic acid, wheat protein, hair keratin amino acids, or a mixture thereof.

The pharmaceutical composition can be adapted for oral or topical administration. Pharmaceutical compositions adapted for topical administration can further comprise an exfoliant. The exfoliant can be an enzymatic exfoliant, such as papain or bromalein or a mono- or -poly-hydroxy acid. For example, the exfoliant can be an alpha-hydroxy acid, beta-hydroxy acid, or tannic acid. In one embodiment the exfoliant is glycolic acid, lactic acid, citric acid, salicylic acid, or tannic acid. The pharmaceutical composition can include a pharmaceutically acceptable carrier or excipient.

When the pharmaceutical composition includes hydrogen peroxide it may further include an amphoteric surfactant and citric acid in an amount sufficient to inhibit hydrogen peroxide decomposition at 40° C. for at least three months.

The invention is further directed to methods for managing one or more dermatological conditions in a patient which comprises administering to the patient a therapeutically effective amount of a pharmaceutical composition comprising an extract of Morinda citrifolia. The dermatological condition can be one or more of dry skin; dandruff; warts; acne; keratosis; psoriasis; eczema; pruritus; age spots; reduced skin moisture; spider veins; senile purpura; lentigines; melasmas; deepening of skin lines; blotches; wrinkles; blemished skin; nodules; atrophy; rosacea; impetigo; precancerous lesions; elastotic changes; telangiecatic skin; hyperpigmented skin; hyperkeratotic skin; nail infections; sun damaged skin, and inflammatory dermatoses. In one embodiment the dermatological condition is one or more of are wrinkles, aged skin, sun-damaged skin, acne, or psoriasis.

The pharmaceutical composition can administered in an amount of between about 1 mg to 20,000 mg per day and may be administered orally or topically. In one embodiment the extract of Morinda citrifolia is administered with one or more of a moisturizing agent or hydrogen peroxide.

### DETAILED DESCRIPTION OF THE PREFERRED EMBODIMENTS

The present invention is directed to pharmaceutical compositions for the management of dermatological conditions. The pharmaceutical compositions comprise an extract of Morinda citrifolia and at least one of a moisturizing agent or hydrogen peroxide. The present invention is also directed to methods of managing dermatological conditions by administering to a patient a therapeutically effective amount of an extract of Morinda citrifolia. Preferably, the methods of the invention employ the pharmaceutical compositions of the invention.

The term “dermatological conditions,” as used herein, means conditions present any where on the skin caused by aging or extrinsic factors such as sunlight, radiations, air pollution, wind, cold, dampness, heat, chemicals, smoke, and smoking. Dermatological conditions include, but are not limited to, dry skin; dandruff; warts; acne; keratosis; psoriasis; eczema; pruritus; age spots; reduced skin moisture; spider veins; senile purpura; lentigines; melasmas; deepening of skin lines; blotches; wrinkles; blemished skin; nodules; atrophy; rosacea; impetigo; precancerous lesions; elastotic changes characterized by leathery, course, rough, dry and yellowish skin; telangiecatic skin; hyperpigmented skin; hyperkeratotic skin; sun damaged skin, and inflammatory dermatoses. Dermatological conditions further include nail conditions including, but not limited to, nail infections and damage to hair including, but not limited to, hair breakage, weathering damage, thinning of hair, and chemically processed hair. Preferably, the dermatological condition is a condition on the skin, more preferably, the dermatological conditions are wrinkles, aged skin, sun-damaged skin, acne, or psoriasis.

The terms “managing” or “management,” as used herein, include one or more of the prevention, treatment, or modification of a dermatological condition.

In a preferred embodiment of the pharmaceutical composition, the extract of Morinda citrifolia is present in an amount from about 0.01 to 80 weight percent, preferably from about 0.1 to 20 weight percent, and more preferably from about 0.5 to 10 weight percent. The extract of Morinda citrifolia may be obtained from any part of the Morinda citrifolia plant including, for example, the fruit, the skin or rind of the fruit, the seeds, the bark, the leaves, the roots, the flowers, or the stem.

In one embodiment, the pharmaceutical compositions of the invention further includes hydrogen peroxide. The hydrogen peroxide is present in an amount sufficient to cleanse at least a portion of the skin. “Cleanse” as used herein includes the removal of dirt, debris, air pollutants, desquamating cells, and cutaneous secretions of the skin. Preferably, the hydrogen peroxide is present in an amount to cleanse the skin without substantial irritation. The hydrogen peroxide is typically present in an amount from about 0.01 to 6 weight percent, preferably 0.05 to 4 weight percent, and more preferably 0.1 to 1 weight percent of the composition. Without wishing to be bound by theory it is believed that cleansing the skin with hydrogen peroxide improves penetration of the extract of Morinda citrifolia into the skin. Hydrogen peroxide is typically used in topical compositions.

In another embodiment, the pharmaceutical compositions of the invention further includes one or more moisturizing agents. The phrase “moisturizing agent,” as used herein, includes any agent that facilitates hydration of the skin by inhibiting or preventing loss of water from the skin, absorbs water from the atmosphere and hydrates the skin, or enhances the skin's own ability to absorb water directly from the atmosphere, or a combination thereof. Without wishing to be bound by theory it is believed that the moisturizing agent also improves the skins ability to absorb the extract of Morinda citrifolia. Furthermore, moisturizing agents minimize or prevent the skin from drying and cracking; cracked skin is more susceptible to environmental factors that generate free radicals, which are believed to cause further damage to the skin. Suitable moisturizing agents include, but are not limited to, hydrophobic agents, and hydrophilic agents, or combinations thereof. Moisturizers, when used, are typically present in an amount from about 0.01 to 20 weight percent, preferably about 0.05 to 10 weight percent, more preferably from about 0.1 to 5 weight percent of the composition.

Moisturizing agents that are hydrophobic agents include, but are not limited to, ceramide, borage oil (linoleic acid), tocopherol (Vitamin E), tocopherol linoleate, dimethicone, glycerine, and mixtures thereof. Hydrophobic agents, when present, are believed to moisturize the skin by inhibiting or preventing the loss of water from the skin. The hydrophobic agent, when present, is typically present in an amount from about 0.01 to 20 weight percent, preferably from about 0.05 to 15 weight percent, and more preferably from about 0.1 to 5 weight percent of the composition.

Moisturizing agents that are hydrophilic agents include, but are not limited to, hyaluronic acid, sodium peroxylinecarbolic acid (sodium PCA), wheat protein (e.g., laurdimonium hydroxypropyl hydrolyzed wheat protein), hair keratin amino acids, and mixtures thereof. Sodium chloride may also be present, particularly when hair keratin amino acids are included as a moisturizer. Hydrophilic agents, when present, are believed to moisturize the skin by absorbing moisture from the atmosphere to hydrate or facilitate hydration of the skin. The hydrophilic agent, when present, is typically present in an amount from about 0.01 to 20 weight percent, preferably from about 0.05 to 15 weight percent, and more preferably from about 0.1 to 5 weight percent of the composition.

More preferably, the compositions of the invention include both hydrogen peroxide and a moisturizing agent.

Other moisturizing agents that hydrate the skin and are useful in the compositions and methods of the present invention include, but are not limited to, panthenol; primrose oil; GLA 3 and other fish oils that may include, for example, the omega-3 and omega-6 oils and/or linoleic acid; and flax seed oil. Preferably, these moisturizing agents are administered orally.

The pharmaceutical compositions of the invention may further include a variety of other components. For example, the dermatological compositions further includes an exfoliant to help remove dead or dying skin cells and further improve the skin's own ability to absorb moisture directly from the atmosphere. Preferably, the exfoliant is used in combination with one or more hydrophilic agents to help absorb moisture from the atmosphere and hydrate the skin or in combination with one or more a hydrophobic agents to inhibit or prevent moisture loss by the skin. More preferably, the pharmaceutical composition includes one or more of a hydrophilic agent and one or more of a hydrophobic agent in combination with an exfoliant. It is believed that the exfoliant also helps the extract of Morinda citrifolia penetrate the skin. Exfoliants are typically used in compositions adapted for topical administration.

The exfoliant may be an enzymatic exfoliant, or an acidic exfoliant. Any enzymatic exfoliant known to those skilled in the art may be used in the compositions and methods of the invention. Examples of enzymatic exfoliants useful in the compositions and methods of the invention include, but are not limited to, papain, from papaya, and bromalein, from pineapple.

Examples of acidic exfoliants include, but are not limited to a mono- or poly-hydroxy acid, tannic acid, or a mixture thereof, or a pharmaceutically acceptable salt or ester thereof. One of ordinary skill in the art will be readily able to select and prepare suitable mono- or poly-hydroxy acids for use in the composition of the invention, for example, alkyl hydroxycarboxylic acids, aralkyl and aryl hydroxycarboxylic acids, polyhydroxy-carboxylic acids, and hydroxy-polycarboxylic acids or esters thereof. One of ordinary skill in the art would typically select one or more of the following mono- or poly-hydroxy acids: 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof; and 2-hydroxyphenyl pyruvic acid and esters thereof.

In one embodiment the poly-hydroxy acidic components is an alpha-hydroxy acid. Preferred alpha-hydroxy acids include citric acid, glycolic acid, and lactic acid. In another embodiment the poly-hydroxy acidic exfoliant is a beta-hydroxy acid. A preferred beta-hydroxy acid is salicylic acid.

The term “pharmaceutically acceptable salt” refers to a salt prepared from pharmaceutically acceptable non-toxic acid. Examples of suitable inorganic metallic cations for salts formation with the acid compounds of the invention include, but are not limited to, aluminum, calcium, lithium, magnesium, potassium, sodium, and zinc. Appropriate organic bases for salt formation with the acid compounds of the invention may be selected, for example, from N,N-dibenzylethylenediamine, chloroprocaine, choline, diethanolamine, ethylenediamine, meglumaine (N-methylglucamine), and procaine.

It should be understood that one or more derivatives of the above acidic component, such as esters or lactones thereof, are also suitably used. One of ordinary skill in the art will also understand that various hydroxy acids described in U.S. Pat. Nos. 5,547,988 and 5,422,370 are also suitable for use in the compositions and methods ofthe invention. The acidic component is present in the composition and methods in an amount sufficient to exfoliate, i.e., remove dead or dying skin cells, from at least a portion of the skin. The acidic component is typically present in an amount from about 0.1 to 12 weight percent, preferably about 1 to 11 weight percent, more preferably from about 4 to 10 weight percent of the composition. For example, the acidic component may be from about 0.1 to 3 weight percent citric acid in combination with up to about 2 weight percent salicylic acid.

The pharmaceutical compositions of the invention may further include one or more of a vitamin A source including retinyl palmitate or other retinyl esters, retinoic acid, Retinol, or tazarotene. The Retinol facilitates normal skin production, particularly epidermal normalization, and, when used, is typically present in an amount from about 0.01 to 6 weight percent, preferably about 0.1 to 5 weight percent.

Optionally, the pharmaceutical compositions also include at least one herb from the group of yellow dock, bupleurum, poria cocos, gentian root, myrr gum, hawthorn berry extract, rosemary extract, wild yam root, wild yam extract, marshmallow root, black cohosh, soy, or ginger.

Yellow Dock, also known as Rumex crispus, is often used to treat skin disease, especially those involving some form of inflammation. The active constituents of yellow dock are believed to be rumicin and chrysarobin. Yellow Dock extract, when included, is typically present in the pharmaceutical compositions of the invention in an amount from about 1 to 30 weight percent, preferably from about 3 to 25 weight percent, and more preferably from about 5 to 20 weight percent.

Bupleurum, also known as Bupleurum falactum, is known for its effect on the liver. The active constituents in bupleurum are believed to be furfurol, sterol, and bupleurumol. The bupleurum, when included, is typically present in the pharmaceutical compositions of the invention in an amount from about 1 to 20 weight percent weight, preferably about 2 to 15 weight percent, and more preferably from about 3 to 10 weight percent.

The active constituents in poria cocos, also known as Lycoperdon solidum, are tetracyclic titerpenic acids, polysaccharides, ergostol, choline, lipase, and protease. This herb is useful for reducing or eliminating excess fluids from the body. When included in the pharmaceutical compositions of the invention, it is typically present in an amount from about 1 to 20 weight percent, preferably from about 2 to 15 weight percent, and more preferably from about 3 to 10 weight percent.

The bitter glycosides in gentian root, also known as Gentian lutea, account for its use as a digestive bitter and liver disorder treatment. Gentian root, when included in the pharmaceutical compositions of the invention, is typically present in an amount from about 1 to 20 weight percent, preferably from about 2 to 15 weight percent, and more preferably from about 3 to 10 weight percent.

Myrrh, also known as Commiphora myrrha, has several oils, resins and gums that increase circulation and heart rate. Myrrh gum, when included in the pharmaceutical compositions of the invention, is typically present in an amount from about 1 to 20 weight percent, preferably from about 2 to 15 weight percent, and more preferably from about 3 to 10 weight percent.

Hawthorn berry extract, also known as Crataegus supplement, can optionally be added to the dermatological compositions, as well. This herb is useful in the treatment of heart disease. Crategolic acid, citric acid, tartaric acid, glavone, glycosides, and vitamin C are the active constituents of hawthorne berries. The hawthorn berry extract, when included in the pharmaceutical compositions of the invention, is typically present in an amount from about 0.5 to 8 weight percent, preferably from about 0.6 to 6 weight percent, and more preferably from about 0.7 to 4 weight percent of the composition.

Rosemary contains aromatic oils that my assist with stomach disorders, and salicylic acid. When included in the pharmaceutical compositions of the invention, rosemary is typically present in an amount from about 0.5 to 8 weight percent, preferably from about 0.6 to 6 weight percent, and more preferably from about 0.7 to 4 weight percent of the composition.

Wild yam possesses glycoside saponins and diosgenins, hormonal precursors to cortical steroids that may help to reduce pain. It is believed to assist with problems of the liver and gall bladder, as well. When included in the pharmaceutical compositions of the invention, wild yam is typically present in an amount from about 0.5 to 8 weight percent, preferably from about 0.6 to 6 weight percent, and more preferably from about 0.7 to 4 weight percent.

The marshmallow root, also known as Althea officinalis, acts as an anti-inflammatory. The mucilage in the herb soothes membranes, thereby reducing inflammation. When included in the pharmaceutical compositions of the invention, marshmallow root is typically present in an amount from about 0.5 to 8 weight percent, preferably from about 0.6 to 6 weight percent, and more preferably from about 0.7 to 4 weight percent of the composition.

Black cohosh acts as a natural estrogen supplement. Soy and ginger may act as an anti-oxidant and may act as a moisturizer to hydrate or facilitate hydration of the skin. The amount of these herbs, when present in the pharmaceutical compositions of the invention, may be readily determined by one of ordinary skill in the art.

The pharmaceutical composition may also preferably include one or more anti-inflammatory components in an amount sufficient to reduce redness and swelling of the skin, an immuno-enhancer component in an amount sufficient to boost the immune system to facilitate repair of damaged skin, and one or more additional antioxidants in an amount sufficient to neutralize free radicals, or a combination thereof.

The one or more anti-inflammatory agents is present in an amount sufficient to reduce inflammation of the skin. In one embodiment the anti-inflammatory agent is a steroidal anti-inflammatory. Suitable steroidal anti-inflammatory agents for use in the compositions and methods of the invention include the corticosteroids such as, but not limited to, hydrocortisone, fluocinolone acetonide, halcinonide, halobetasol propionate, clobetasol propionate, betamethasone dipropionate, betamethasone valerate, and triamcinolone acetonide.

In another embodiment the anti-inflammatory agent is a non-steroidal anti-inflammatory agent. Examples of suitable non-steroidal anti-inflammatory agents for use in the compositions and methods of the invention include, but are not limited to, aspirin, ibuprofen, ketoprofen, and naproxen. These anti-inflammatory agents are preferably administered orally. Othernon-steroidal anti-inflammatory agents useful in the compositions of the invention include, but are not limited to, aloe vera gel, aloe vera, licorice extract, pilewort, Canadian willow root, zinc, and allantoin. Allantoin is a preferred non-steroidal anti-inflammatory agent. The anti-inflammatory agents are used in an amount sufficient to inhibit or reduce inflammation, preferably in an amount from about 0.02 to 2 weight percent, preferably from about 0.1 to 1.5 weight percent, and more preferably from about 0.2 to 1 weight percent of the composition. It should be understood, with reference to managing skin conditions, that the anti-inflammatory agents facilitate inhibition or suppression of inflammation any where on the skin. Arnica Montana (a healing herb) and vitamin K can also be used as the anti-inflammatory. Arnica Montana facilitates skin healing and acts as an antiseptic and local anti-inflammatory, and, when used, is typically present in an amount from about 0.1 to 2 weight percent, preferably about 0.2 to 1 weight percent. Vitamin K inhibits or suppresses inflammation and bruising (i.e., acts as an anti-inflammatory and anti-bruising agent) and, when used, is typically present in an amount from about 0.01 to 1 weight percent, preferably from about 0.1 to 0.5 weight percent.

The immuno-enhancer component is present in an amount sufficient to boost the immune system to facilitate repair of damaged skin. Suitable immuno-enhancers useful in the compositions of the invention include, but are not limited to, interferon, Aldara (Immiquimod), resiquimod, and tacrolimus (Prograf). The immuno-enhancer may be present in an amount from about 0.1 to 10 weight percent, preferably from about 0.5 to 5 weight percent of the composition.

The invention also contemplates using stem-cell therapy to improve the skins ability to function. In one embodiment of the invention, stem-cells, preferably from the patients own body, are contacted with the patients skin, typically by injection. The stem cells then develop into new skin cells and, thus, improve the skins ability to function.

Anti-oxidants of both the enzymatic and non-enzymatic type may be included in the compositions and methods of the invention. For example, superoxide dismutase (SOD), catalase, and glutathione peroxidase are natural enzymatic anti-oxidants used by the body that may be supplemented with the compositions herein. Suitable non-enzymatic anti-oxidants include, but are not limited to, Vitamin E (e.g., tocopherol), Vitamin C (ascorbic acid), carotenoids, Echinacoside and caffeoyl derivatives, oligomeric proanthocyanidins or proanthanols (e.g., grape seed extract), silymarin (e.g., milk thistle extract, Silybum marianum), ginkgo biloba, green tea polyphenols, pomegranate extract, and mixtures thereof. Carotenoids are powerful anti-oxidants, and they include beta-carotene, canthaxanthin, zeaxanthin, lycopen, lutein, crocetin, capsanthin, and the like. Preferably, the anti-oxidant component includes Vitamin E, Vitamin C, or a carotenoid. When vitamin C component is used as an antioxidant it is preferably an ascorbic acid, or a pharmaceutically acceptable salt or ester thereof, and more preferably ascorbyl palmitate, dipalmitate L-ascorbate, sodium L-ascorbate-2-sulfate, or an ascorbic salt, such as sodium, potassium, and calcium, or mixtures thereof. When oral formulations of the pharmaceutical composition are used, it is preferred that a non-acidic form of vitamin C be used to reduce the stomach irritation that may occur when using an acidic form. The anti-oxidant component, when used, is present in an amount sufficient to inhibit or reduce the effects offree-radicals. The anti-oxidant component maybe present in an amount from about 0.001 to 1 weight percent, preferably from about 0.01 to 0.5 weight percent of the composition.

In another embodiment, the pharmaceutical compositions further comprise a pharmnaceutically acceptable antimicrobial agent. Any pharmaceutically acceptable antimicrobial agent available to those of ordinary skill in the art may be used, but preferably at least one of an antibacterial agent, antifungal agent, antiviral agent, or anthelmintic will be used according to the invention. A single broad spectrum antimicrobial agent, i.e., one that is believed to have at least two of antibacterial, antifungal, and antiviral efficacy, including, but not limited to echinacea, golden seal, benzalkonium chloride, benzethonium chloride, iodine, grape seed extract, pomegranate extract, green tea extract or polyphenols, or combinations thereof, may be included. Another suitable antimicrobial agent includes the class of anthelmintics, such as metronidazole, to facilitate treatment of, e.g., tricomona infection. Preferred antiviral agents include, but are not limited to, acyclovir, tamvir, penciclovir, and the like, and mixtures thereof. Preferred antibacterial agents include, but are not limited to, triclosan, neomycin, polymyxin, bacitracin, clindamycin, benzoyl peroxide, a tetracycline, a sulfa drug, a penicillin, a quinolone, a cephalosporin, and mixtures thereof. Preferred antifungal agents include, but are not limited to, famesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and the like, and mixtures thereof. Exemplary tetracyclines include doxycycline and minocycline. An exemplary sulfa drug is sulfacetamide. An exemplary cephalosporin is cephalexin (commercially available as KEFLEX). Exemplary quinolones include the floxacins, such as loemfloxacin, ofloxacin, and trovafloxacin. It should be readily understood that any salts, isomers, pro-drugs, metabolites, or other derivatives of these antimicrobial agents may also be included as the antimicrobial agent in accordance with the invention. The antimicrobial agent is typically present in an amount from about 0.01 to 1.5 weight percent, preferably from about 0.1 to 1.2 weight percent, and more preferably from about 0.3 to 1 weight percent of the composition. The antimicrobial agent inhibits the formation of, and may further reduce the presence of, microbes that cause redness, inflammation, and irritation of the skin.

The pharmaceutical composition may further optionally include one or more of a cysteine component, magnesium component, manganese component, carotenoid component, selenium component, and copper component.

The optional cysteine component assists in thickening the dermis, supplementing of collagen and elastic tissue, and consequently, reduction of wrinkles and other skin conditions. The cysteine component, when used in the composition, is preferably N-acetyl cysteine, or a pharmaceutically acceptable salt thereof, and is then typically present in an amount from about 1 to 10 weight percent, preferably from about 2 to 8 weight percent, and more preferably from about 3 to 6 weight percent of the composition.

The optional manganese component is the co-factor used by the SOD found in mitochondria. The manganese component may be any manganese compound, or pharmaceutically acceptable salt thereof, but preferably is manganese ascorbate or a manganese ascorbic acid complex. The manganese, when present, is typically present in an amount from about 0.5 to 10 weight percent, preferably from about 1 to 8 weight percent and most preferably from about 5 to 7 weight percent, wherein the manganese is present in an amount from about 5 to 20 weight percent of a complex such as manganese ascorbate.

The copper component may also be included in the pharmaceutical composition, and may be any copper compound, or a pharmaceutically acceptable salt thereof. The copper component inhibits elastase and assists in treatment of elastic tissue defects. Preferably, the copper compound is copper sebacate. The copper, when included in the composition, is typically present in an amount from about 5 to 20 weight percent of the copper sebacate. The copper component is typically present in an amount from about 0.01 to 5 weight percent, preferably from about 0.02 to 3 weight percent, and more preferably from about 0.03 to 2 weight percent of the composition.

The magnesium component is also optional and may be any magnesium compound, or a pharmaceutically acceptable salt thereof, but preferably is magnesium ascorbate or magnesium ascorbic acid complex, wherein the magnesium is typically present in about 5 to 20 weight percent of the complex. The magnesium component, when included in the composition, is typically present in an amount from about 1 to 10 weight percent, preferably from about 3 to 8 weight percent, and more preferably from about 5 to 7 percent of the composition.

Additionally, a source of selenium may also be optionally added to the pharmaceutical composition. A selenium compound, or a pharmaceutically acceptable salt thereof, may be used. When present, the selenium compound is preferably selenium complexed with an amino acid. More preferably, the selenium compound is L-selenomethionine, wherein the selenium is present in an amount from about 0.1 to 5 weight percent of the complex. The selenium, when included, is typically present in an amount from about 0.01 to 3 weight percent, preferably from about 0.05 to 2 weight percent, and more preferably from about 0.1 to 1 weight percent in the pharmaceutical composition.

The pharmaceutical compositions of the invention may also include one or more of a local analgesic or anesthetic, antiyeast agent, antiperspirant, antipsoriatic agent, antiaging agent, antiwrinkle agent, sun screen and/or sun blocking agent, skin lightening agent, depigmenting agent, vitamin, hormone, or retinoid.

The compositions of the invention may further include one or more surfactants, stabilizers, preservatives, coloring agents, water, buffering agents, emulsifying agents, thickeners, solvents, perfuming agents, and the like. Preferably, the water is deionized water. It should be understood that water includes the remainder of a given composition after other ingredients are determined. Although any pharmaceutically acceptable surfactant, stabilizer, preservative, coloring agent, buffering agents, emulsifying agents, thickeners, solvents, or perfuming agents may be used, certain compounds or mixtures are preferred as discussed below.

Preferred surfactants, including both the foaming and non-foaming type, including, but not limited to, sodium laureth sulfate, sodium laureth-13 carboxylate, disodium laureth sulfosuccinate, disodium cocoamphodiacetate, and the like, and mixtures thereof. More preferably, at least one amphoteric surfactant is included in the composition, such as disodium cocoamphodiacetate. The amphoteric surfactant, in combination with citric acid, inhibits hydrogen peroxide decomposition. The surfactant component maybe present in an amount from about 10 to 90 weight percent, preferably about 20 to 80, and more preferably about 30 to 70 weight percent of the composition.

The term “inhibit hydrogen peroxide decomposition,” as used herein, means to at least stop the rate of decomposition from increasing, preferably to inhibit the decomposition entirely, and more preferably to substantially inhibit the decomposition altogether. “Substantially inhibit,” as used herein, means that less than about 10 weight percent, preferably less than about 3 weight percent, and more preferably less than about 1 weight percent, of the hydrogen peroxide decomposes over a three month period of time at 40° C.

A preferred stabilizer includes glycol stearate or PEG-150 distearate. The stabilizer, when used, is typically present in an amount from about 0.1 to 5 weight percent of the composition.

Preferred preservatives include tetrasodium ethylene-diamine tetraacetic acid (EDTA), methylparaben, benzophenone-4, methylchloroisothiazolinone, methylisothiazolinone, and the like, and mixtures thereof. Preservatives, when used, are typically present in an amount from about 0.01 to 6 weight percent, preferably about 0.05 to 4 weight percent, and more preferably from about 0.1 to 2 weight percent.

Preferred coloring agents include FD&C Green No. 3, FD&C Violet No. 2, FD&C Yellow No. 5, FD&C Red No. 40, and the like, and mixtures thereof. The coloring agents, when used, are typically present in an amount from about 0.001 to 0.1 weight percent, and preferably from about 0.005 to 0.05 weight percent of the composition.

The pharmaceutical compositions of the invention may also include a pharmaceutically acceptable carrier. Any suitable pharmaceutically acceptable carrier readily apparent to those of ordinary skill in the art may be combined with the extract of Morinda citrifolia and the at least one of a moisturizing agent or hydrogen peroxide, to provide the pharmaceutical compositions of the invention. Pharmaceutically acceptable carriers include, but are not limited to, hydroxypropyl cellulose, starch (corn, potato, rice, wheat), pregelatinized starch, gelatin, sucrose, acacia, alginic acid, sodium alginate, guar gum, ethyl cellulose, carboxymethylcellulose sodium, carboxymethylcellulose calcium, polyvinylpyrrolidone, methylcellulose, hydroxyproplymethylcellulose, microcrystalline cellulose, polyethylene glycol, powdered cellulose, glucose, croscarmellose sodium, crospovidone, polacrilinpotassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerin, sorbitol, mannitol, stearic acid, sodium lauryl sulfate, hydrogenated vegetable oil (e.g., peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones. For example, preferred topical formulations of the pharmaceutical composition may include a silicon-containing carrier, but in amounts insufficient to cause substantial irritation. Suitable silicones include cyclomethicone or a mixture of cyclopentasiloxane and dimethicone/vinyldimethicone crosspolymer.

The ranges of the components of the pharmaceutical composition may vary, but the active ingredients should be understood to add to 100 weight percent of the active pharmaceutical composition.

The pharmaceutical compositions of the invention may be adapted for any route of administration including, but not limited to, topical, oral, rectal, parenteral, intravenous, transdermal, subcutaneous, and intramuscular. Preferably the pharmaceutical compositions of the invention are adapted for topical or oral administration, with topical administration being most preferred, it being understood that some of the components may not be suitable for various modes of administration. One of ordinary skill in the art would readily realize what components are suitable with each mode of administration. Topical compositions may be prepared in high concentrations for administration to be removed shortly thereafter, as well as in lower concentrations that are safer for products that can remain in contact with the skin for longer times.

The present invention is further directed to a method of managing one or more dermatological conditions. The methods of the invention comprise administering to a patient in need thereof a therapeutically effective amount of an extract of Morinda citrifolia.

The term “therapeutically effective amount,” as used herein, means that amount of the pharmaceutical composition that provides a therapeutic benefit in the management of one or more dermatological conditions.

Any suitable route of administration may be employed for providing the patient with a therapeutically effective amount of an extract of Morinda citrifolia, including, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation and like forms of administration. Preferably, the an extract of Morinda citrifolia is administered orally or topically, with topical administration being most preferred. Typically, the an extract of Morinda citrifolia is administered as a pharmaceutical composition comprising a pharmaceutically acceptable excipient. Optionally, the extract of Morinda citrifolia is administered in conjunction with one or more other dermatological components, such as those described above. The one or more other dermatological components may be administered in conjunction, i.e., concurrently or sequentially, with the extract of Morinda citrifolia. Preferably, the extract of Morinda citrifolia is administered as a pharmaceutical composition of the invention.

The magnitude of a prophylactic or therapeutic dose of the extract of Morinda citrifolia in managing a dermatological condition will vary with the sensitivity of the person's skin and the route of administration. The dose, and perhaps the dose frequency, will also vary according to the age, body weight, and response of the individual patient. In general, the total daily dose range, for the conditions described herein, is from about 1 mg to about 2,000 mg administered in about one to ten doses, preferably two to eight doses. The preferred oral daily dose range should be from about 1 mg to 2,000 mg, more preferably from about 400 mg to 1,600 mg, and most preferably from about 800 mg to 1,200 mg. In general, a preferred topical daily dosage range, in single or divided doses, should be from about 1 mg to 20,000 mg, more preferably from about 2,000 mg to 16,000 mg, and most preferably from about 6,000 mg to 10,000 mg of the compositions.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient's response.

Dosage forms for use in the methods of the present invention suitable for oral administration include, but are not limited to, suspensions, solutions, elixirs, and aerosols; and may include carriers, such as starches, sugars, microcrystalline cellulose, diluents, granulating agents, lubricants, binders, disintegrating agents, and the like. In the case of oral solid preparations (such as powders, capsules, and tablets), the oral solid preparations are typically preferred over the oral liquid preparations.

Suitable dosage forms for topical administration include, but are not limited to, dispersions, lotions; creams; gels; pastes; powders; aerosol sprays; syrups or ointments on sponges or cotton applicators; and solutions or suspensions in an aqueous liquid, non-aqueous liquid, oil-in-water emulsion, or water-in-oil liquid emulsion. Because of its ease of administration, a cream, lotion, or ointment represents the most advantageous topical dosage unit form, in which case liquid pharmaceutical carriers maybe employed in the composition. These creams, lotions, or ointments, may be prepared as rinse-off or leave-on products, as well as two stage treatment products for use with other skin cleansing or managing compositions. In a preferred embodiment, the compositions are administered as a rinse-off product in a higher concentration form, such as a gel, and then a leave-on product in a lower concentration to avoid irritation of the skin. Each of these forms is well understood by those of ordinary skill in the art, such that dosages may be easily prepared to incorporate the pharmaceutical composition of the invention.

The pharmaceutical compositions of the invention maybe prepared by any of the methods of pharmacy, but all methods include the step of bringing into association the carrier(s) with the active ingredient, which constitutes one or more necessary ingredients. In general, the compositions are prepared by uniformly and intimately admixing the active ingredient with liquid carriers or finely divided solid carriers or both, and then, if necessary, shaping the product into the desired presentation.

Various modifications of the invention in addition to those described herein will be apparent to those skilled in the art from the foregoing description. Such modifications are also intended to fall within the scope of the appended claims. The foregoing disclosure includes all the information deemed essential to enable those skilled in the art to practice the claimed invention.

What is claimed is:

1. A pharmaceutical composition for managing a dermatological condition comprising an extract of Morinda citrifolia and a moisturizing agent in an amount sufficient to facilitate hydration of the skin.

2. The composition of claim 1, wherein the extract of Morinda citrifolia is present in an amount of from about 0.01 to 80 percent byweight of the composition and the moisturizing agent is present in an amount of about 0.01 to 20 percent by weight of the composition.

3. The pharmaceutical composition of claim 1, wherein the moisturizing agent is a hydrophobic moisturizing agent.

4. The composition of claim 3, wherein the hydrophobic moisturizing agent is ceramide, borage oil, tocopherol, tocopherol linoleate, dimethicone, glycerine, or a mixture thereof.

5. The pharmaceutical composition of claim 1, wherein the moisturizing agent is a hydrophilic moisturizing agent.

6. The pharmaceutical composition of claim 5, wherein the hydrophilic moisturizing agent is hyaluronic acid, sodium peroxylinecarbolic acid, wheat protein, hair keratin amino acids, or a mixture thereof.

7. The pharmaceutical composition of claim 1 adapted for topical administration.

8. The pharmaceutical composition of claim 1 adapted for oral administration.

9. The pharmaceutical composition of claim 7, further comprising an exfoliant.

10. The pharmaceutical composition of claim 9, wherein the exfoliant is an enzymatic exfoliant.

11. The pharmaceutical composition of claim 10, wherein the enzymatic exfoliant is papain or bromalein.

12. The pharmaceutical composition of claim 9, wherein the exfoliant is an mono- or -poly-hydroxy acid.

13. The pharmaceutical composition of claim 12, wherein the exfoliant comprises an alpha-hydroxy acid, beta-hydroxy acid, or tannic acid.

14. The pharmaceutical composition of claim 13, wherein the exfoliant comprises glycolic acid, lactic acid, citric acid, salicylic acid, or tannic acid.

15. The pharmaceutical composition of claim 1, wherein the composition further comprises a pharmaceutically acceptable carrier or excipient.

16. The pharmaceutical composition of claim 7, further comprising hydrogen peroxide in an amount sufficient to cleanse the skin.

17. The pharmaceutical composition of claim 16, wherein the hydrogen peroxide is present in an amount of from about 0.01 to 6 percent by weight of the composition.

18. The pharmaceutical composition of claim 16, further comprising an amphoteric surfactant and citric acid in an amount sufficient to inhibit hydrogen peroxide decomposition at 40° C. for at least three months.

19. The pharmaceutical composition of claim 16, wherein the moisturizing agent is a hydrophobic moisturizing agent.

20. The composition of claim 19, wherein the hydrophobic moisturizing agent is ceramide, borage oil, tocopherol, tocopherol linoleate, dimethicone, glycerine, or a mixture thereof.

21. The pharmaceutical composition of claim 16, wherein the moisturizing agent is a hydrophilic moisturizing agent.

22. The pharmaceutical composition of claim 21, wherein the hydrophilic moisturizing agent is hyaluronic acid, sodium peroxylinecarbolic acid, wheat protein, hair keratin amino acids, or a mixture thereof.

23. The pharmaceutical composition of claim 16, further comprising an exfoliant.

24. The pharmaceutical composition of claim 23, wherein the exfoliant is an enzymatic exfoliant.

25. The pharmaceutical composition of claim 24, wherein the enzymatic exfoliant is papain or bromalein.

26. The pharmaceutical composition of claim 23, wherein the exfoliant is an mono- or -poly-hydroxy acid.

27. The pharmaceutical composition of claim 26, wherein the exfoliant comprises an alpha-hydroxy acid, beta-hydroxy acid, or tannic acid.

28. The pharmaceutical composition of claim 27, wherein the exfoliant comprises glycolic acid, lactic acid, citric acid, salicylic acid, or tannic acid.

29. The pharmaceutical composition of claim 16, wherein the composition further comprises a pharmaceutically acceptable carrier or excipient.

30. A topical pharmaceutical composition for managing a dermatological condition comprising an extract of Morinda citrifolia and hydrogen peroxide in an amount sufficient to cleanse the skin.

31. The composition of claim 30, wherein the extract of Morinda citrifolia is present in an amount of from about 0.01 to 80 percent by weight of the composition and the hydrogen peroxide is present in an amount of about 0.01 to 6 percent by weight of the composition.

32. The pharmaceutical composition of claim 30, further comprising an amphoteric surfactant and citric acid in an amount sufficient to inhibit hydrogen peroxide decomposition at 40° C. for at least three months.

33. The pharmaceutical composition of claim 30, further comprising an exfoliant.

34. The pharmaceutical composition of claim 33, wherein the exfoliant is an enzymatic exfoliant.

35. The pharmaceutical composition of claim 34, wherein the enzymatic exfoliant is papain or bromalein.

36. The pharmaceutical composition of claim 33, wherein the exfoliant is an mono- or -poly-hydroxy acid.

37. The pharmaceutical composition of claim 36, wherein the exfoliant comprises an alpha-hydroxy acid, beta-hydroxy acid, or tannic acid.

38. The pharmaceutical composition of claim 37, wherein the exfoliant comprises glycolic acid, lactic acid, citric acid, salicylic acid, or tannic acid.

39. The pharmaceutical composition of claim 30, wherein the composition further comprises a pharmaceutically acceptable carrier or excipient.

40. A method for managing one or more dermatological conditions in a patient which comprises administering to the patient a therapeutically effective amount of a pharmaceutical composition comprising an extract of Morinda citrifolia.

41. The method of claim 40, wherein the dermatological condition is one or more of dry skin; dandruff; warts; acne; keratosis; psoriasis; eczema; pruritus; age spots; reduced skin moisture; spider veins; senile purpura; lentigines; melasmas; deepening of skin lines; blotches; wrinkles; blemished skin; nodules; atrophy; rosacea; impetigo; precancerous lesions; elastotic changes; telangiecatic skin; hyperpigmented skin; hyperkeratotic skin; nail infections; sun damaged skin, and inflammatory dermatoses.

42. The method of claim 41, wherein the dermatological condition is one or more of are wrinkles, aged skin, sun-damaged skin, acne, or psoriasis.

43. The method of claim 40, wherein the pharmaceutical composition is administered in an amount of between about 1 mg to 20,000 mg per day.

44. The method of claim 43, wherein the dermatological agent is administered orally.

45. The method of claim 43, wherein the dermatological agent is administered topically.

46. The method of claim 45, further comprising topically administering one or more of a moisturizing agent or hydrogen peroxide.

47. The method of claim 46, wherein the moisturizing agent is a hydrophobic moisturizing agent.

48. The method of claim 47, wherein the hydrophobic moisturizing agent is ceramide, borage oil, tocopherol, tocopherol linoleate, dimethicone, glycerine, or a mixture thereof.

49. The method of claim 46, wherein the moisturizing agent is a hydrophilic moisturizing agent.

50. The method of claim 49, wherein the hydrophilic moisturizing agent is hyaluronic acid, sodium peroxylinecarbolic acid, wheat protein, hair keratin amino acids, or a mixture thereof.

51. The method of claim 45, further comprising topically administering an exfoliant.
